(19) United States
(12) Patent Application Publication
(10) Pub. No.: US 2008/0300320 A1
(43) Pub. Date: Dec. 4, 2008

(54) Use of Polyvinyl Lactam-Polyalkylene Block Copolymers as Solubilisers for Poorly Water-Soluble Compounds

(75) Inventors: Ronald Frans Maria Lange, Ludwigshafen (DE); Nathalie Bouillo, Baden-Baden (DE); Ingo Munster, Bohl-Iggelheim (DE); Klemens Mathauer, Taipei (TW); Kathrin Meyer-Bohm, Feucht (DE); Rainer Dobrawa, Mannheim (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/096,544

(22) PCT Filed: Nov. 30, 2006
(86) PCT No.: PCT/EP2006/069129

§ 371 Date: Jun. 6, 2008

(30) Foreign Application Priority Data
Dec. 9, 2005 (EP) 05111923.8

(51) Int. Cl. A61K 47/30; A61K 8/90; A01N 25/00; A23L 1/00

(52) U.S. Cl. 514/772.1; 504/360; 426/531

(57) ABSTRACT

Use of polyvinyllactam-polyalkylene oxide block copolymers as solubilizers for sub-stances which are sparingly soluble in water.

The invention relates to the use of polyvinyllactam-polyoxyalkylene block copolymers as solubilizers of biologically active substances which are sparingly soluble in water. In addition, the invention relates to corresponding preparations for use on humans, animals and plants.

When producing homogeneous preparations of biologically active substances, the solubilization of hydrophobic substances, i.e. substances which are sparingly soluble in water, has achieved very great practical importance.

Solubilization is understood as meaning making substances which are insoluble or sparingly soluble in a certain solvent, in particular water, soluble through interface-active compounds, the solubilizers. Such solubilizers are able to convert poorly water-soluble or water-insoluble substances into clear, at most opalescent aqueous solutions without the chemical structure of these substances undergoing a change in the process (cf. Römpp Chemie Lexikon, 9^{th} edition, Vol. 5, p. 4203, Thieme Verlag, Stuttgart, 1992).

The prepared solubilizates are notable for the fact that the poorly water-soluble or water-insoluble substance is present in colloidally dissolved form in the molecular associates of the surface-active compounds which form in aqueous solution—the so-called micelles. The resulting solutions are stable single-phase systems which appear to be visually clear to opalescent and can be prepared without the input of energy.

Solubilizers can, for example, improve the appearance of cosmetic formulations and of food preparations by making the formulations transparent. Furthermore, in the case of pharmaceutical preparations, the bioavailability and thus the effect of medicaments can also be increased through the use of solubilizers.

The solubilizers used for pharmaceutical medicaments and cosmetic active ingredients are primarily surfactants such as ethoxylated (hydrogenated) castor oil, ethoxylated sorbitan fatty acid esters or ethoxylated hydroxystearic acid. However, the hitherto used solubilizers described above have a number of application-related disadvantages.

The known solubilizers have only a small solubilizing effect for some sparingly soluble medicaments such as, for example, clotrimazole.

In addition, the solubilizers known hitherto are mostly liquid or semisolid compounds which, on account of this, have relatively unfavorable processing properties.

WO 94/20073 discloses polyvinylpyrrolidone block copolymers and their use as wall material for liposomes.

WO 03/072158 discloses polyvinylpyrrolidone block copolymers and their use in medicinal technology, for example for the surface modification of catheters. For the production of the block copolymers, the hydroxyl-functionalization of polyvinylpyrrolidone with isopropoxyethanol is described.

The object was to provide novel solubilizers for pharmaceutical, cosmetic, food and agrotechnical applications.

According to the invention, the object was achieved through the use of polyvinyllactam-polyalkylene oxide block copolymers as solubilizers for substances which are sparingly soluble in water.

In the text below, the polyvinyllactam block is referred to as A block, and the polyalkylene oxide block is referred to as B block.

In order to enable the polyalkylene block to couple onto the polyvinyllactam block, the polyvinyllactams are functionalized with hydroxyl groups at the start of the chain and/or at the end of the chain. The OH functionalization can be achieved either via the free-radical initiator or via a regulator. Functionalization via the free-radical initiator takes place at the start of the chain, functionalization by the regulator at the end of the chain. In order to achieve functionalization, it is therefore necessary to use at least one hydroxyl-carrying free-radical initiator or a hydroxyl-carrying regulator in the polymerization of the polyvinyllactam prepolymers. If B-A-B block copolymers are to be produced, free-radical initiators and regulators must carry hydroxyl groups.

General methods of producing vinyllactam prepolymers are known per se. The production takes place free-radically initiated polymerization in water-miscible solvents or in mixed nonaqueous/aqueous solvents. Suitable N-vinyllactams are N-vinylpyrrolidone, N-vinylcaprolactam or N-vinylpiperidone or mixtures thereof. Preference is given to using N-vinylpyrrolidone.

Suitable nonaqueous solvents are, for example, alcohols, such as methanol, ethanol, n-propanol, and isopropanol, and also glycols, such as ethylene glycol and glycerol.

Further suitable solvents are acetic esters, such as, for example, ethyl acetate or butyl acetate.

The polymerization is preferably carried out at temperatures of from 60 to 100° C.

To initiate the polymerization, free-radical initiators are used. The amounts of initiator or initiator mixtures used, based on the monomer used, are between 0.01 and 10% by weight, preferably between 0.3 and 5% by weight.

Depending on the type of solvent used, either organic or inorganic peroxides, such as sodium persulfate, or azo initiators, such as azobisisobutyronitrile, azobis(2-amido-propane) dihydrochloride or 2,2′-azobis(2-methylbutyronitrile) are suitable.

Peroxidic initiators are, for example, dibenzoyl peroxide, diacetyl peroxide, succinyl peroxide, tert-butyl perpivalate, tert-butyl 2-ethylhexanoate, tertbutyl permaleate, bis-(tert-butylperoxy)cyclohexane, tert-butyl peroxyisopropylcarbonate, tert-butyl peracetate, 2,2-bis(tert-butylperoxy)butane, dicumyl peroxide, di-tert-amyl peroxide, di-tert-butyl peroxide, p-menthane hydroperoxide, pinane hydroperoxide, cumene hydroperoxide, tertbutyl hydroperoxide, hydrogen peroxide, and mixtures of said initiators. Said initiators can also be used in combination with redox components such as ascorbic acid.

If the OH functionalization is to take place via the free-radical initiator, of suitability are, in particular, OH-functionalized initiators such as, for example, 2,2′-azobis[2-methyl-N-(2-hydroxyethyl)propionamide], 2,2′-azobis{2-methyl-N-[2-(1-hydroxybutyl)]propionamide} or 2,2′-azobis{2-[1-(2-hydroxyethyl)-2-imidazolin-2-yl]propane}dihydrochloride.

The free-radical polymerization can, if appropriate, take place in the presence of emulsifiers, if appropriate further protective colloids, if appropriate buffer systems and if appropriate subsequent pH adjustment by means of bases or acids.

Suitable molecular weight regulators are hydrogen sulfide compounds, such as alkyl mercaptans, e.g. n-dodecyl mercaptan, tert-dodecyl mercaptan, thioglycolic acid and esters thereof, mercaptoalkanols, such as mercaptoethanol. Further suitable regulators are specified, for example, in DE 197 12 247 A1, page 4. The required amount of the molecular weight regulator is in the range from 0 to 5% by weight, based on the amount of monomer to be polymerized, in particular 0.05 to 2% by weight, particularly preferably 0.1 to 1.5% by weight. Preference is given to using mercaptoethanol.

The monomer or a monomer mixture or the monomer(s) emulsion are initially introduced together with the initiator, which is usually in the form of a solution, into a stirred reactor at the polymerization temperature (batch process), or, if appropriate, are metered into the polymerization reactor continuously or in several successive stages (feed method). In the feed method, it is customary for the reactor to be filled, prior to the start of the actual polymerization, not only with water (in order to permit stirring of the reactor), but also with partial amounts, seldomly the total amount intended for the polymerization, of the feed materials, such as emulsifiers, protective colloids, monomers, regulators etc. or partial amounts of the feeds (generally monomer or emulsion feed and initiator feed).

Suitable polyalkylene oxides are preferably polyalkylene glycols. The polyalkylene glycols can have molecular weights of from 300 to 25,000 D [daltons], preferably 1,000 to 15,000 D, particularly preferably 1,000 to 10,000 D. The molecular weights are determined starting from the hydroxyl number measured in accordance with DIN 53240.

Suitable particularly preferred polyalkylene glycols are polyethylene glycols. Furthermore, polypropylene glycols, polytetrahydrofurans or polybutylene glycols which are obtained from 2-ethyloxirane or 2,3-dimethyloxirane are also suitable.

Suitable polyethers are also random or blocklike copolymers of polyalkylene glycols obtained from ethylene oxide, propylene oxide and butylene oxide, such as, for example, polyethylene glycol-polypropylene glycol block copolymers. The block copolymers can be of the AB or ABA type.

The preferred polyalkylene glycols also include those which are substituted on one of the two OH end groups. Suitable substituents are alkyl, aryl or aralkyl radicals having 1 to 30 carbon atoms. Suitable aryl radicals are phenyl, naphthyl radicals. Suitable aralkyl radicals are, for example, benzyl radicals. Suitable alkyl radicals are branched or unbranched, open-chain or cyclic C₁- to C₂₂-alkyl radicals. Suitable cycloalkyl radicals are, for example, cyclopentyl, cyclohexyl, cycloheptyl or cyclooctyl radicals which, if appropriate, may be substituted by one or more C1-C4-alkyl radicals. Preferably, C₁-C₁₈-alkyl radicals, for example methyl, ethyl, n-butyl, isobutyl, pentyl, hexyl, octyl, nonyl, decyl, dodecyl, tridecyl or octadecyl radicals, are suitable.

Such polyalkylene oxides are either easy to prepare or commercially available.

The polyalkylene oxides are reacted in equimolar amounts, based on the hydroxyl groups in the polyalkylene oxide and in the vinyllactam prepolymer. The amount of OH groups present can, if required, take place in a manner known per se to the person skilled in the art. To determine the hydroxyl number, see, for example, Römpp Chemie Lexikon, 9th edition, 1990.

Which polyalkylene oxides are used in individual cases is governed by the desired block structure. If a B-A-B type or a A-B type is desired, a polyalkylene oxide can be used whose OH group is substituted at one end. If a AB-A type is desired, it is only possible to use a polyalkylene oxide which has free OH groups at both ends.

The coupling of vinyllactam polymers and polyalkylene oxides take place by reaction with diisocyanates, where reaction with the hydroxyl groups of the vinyllactam copolymer leads to a coupling of the polyalkyleneoxide onto the vinyllactam copolymer via urethane groups. Here, either the vinyllactam polymer or the polyalkylene oxide can firstly be reacted with the diisocyanate.

According to a preferred embodiment of the invention, the coupling takes place via polyalkylene oxides functionalized with isocyanate groups as end groups. For this, firstly the polyalkylene oxide is reacted with the diisocyanate and then the polyalkylene oxide functionalized in this way is reacted with the vinyllactam polymer.

Irrespective of which embodiment is chosen, the reaction can be carried out as follows:

Suitable diisocyanates are compounds of the general formula OCN—R—NCO, where R can be aliphatic, alicyclic or aromatic radicals which may also be substituted by alkyl radicals.

Suitable diisocyanates are preferably compounds whose isocyanate groups have, on account of the molecular structure, a different reactivity toward nucleophiles, for example isophorone diisocyanate or tolylene diisocyanate.

Also suitable in principle are symmetrical diisocyanates, such as, for example, hexamethylene diisocyanate or 4,4′-methylenedi(phenyl isocyanate).

Preference is given to using isophorone diisocyanate.

The reaction with the diisocyanate takes place preferably in an organic solvent, such as ketones, for example acetone, also dimethyl sulfoxide, dimethylformamide, or generally aprotic-polar organic solvents or mixtures of such solvents. The reaction takes place usually at elevated temperatures, the temperature also being governed by the boiling temperature of the solvent chosen. The reaction of the diisocyanate with the first component can take place at 20 to 50° C., but if desired also to 100° C. The reaction of the second isocyanate group can take place at temperatures of from 50 to 100° C.

The reaction preferably takes place equimolarly, which means that the quantitative ratio is chosen so that 1 mol of diisocyanate is used per mole of hydroxyl group to be reacted. If the vinyllactam polymer is OH-functionalized via a regulator, the diisocyanate is reacted equimolarly relative to the regulator. If the vinyllactam polymer is OH-functionalized via a free-radical initiator, then 2 mol of diisocyanate are used per mole of free-radical initiator.

In the case of symmetrical diisocyanates, it may also be advisable to use an excess of diisocyanate and then to remove the excess by distillation.

Preferably, the reaction is carried out in the presence of a catalyst. Suitable catalysts are, for example, organometallic compounds, such as organotitanium compounds or zinc compounds, such as dibutyltin dilaurate or tin octoate, also bases, such as 1,4-diaza(2.2.2)bicyclooctane or tetramethylbutanediamine. The catalyst can be used in amounts of from 0.05 to 0.2 mol, preferably 0.1 to 0.14 mol, per mole of diisocyanate.

The reaction is usually carried out at elevated temperatures in the range from 50 to 100° C. Which temperature is chosen in a specific case depends on the type of organic solvent used. The solvent can then be removed by distillation.

Usually, the reaction is carried out in a way which involves firstly reacting the component, which should be isocyanate-group-functionalized, with the diisocyanate in the presence of the catalyst and a solvent until the isocyanate value in the reaction mixture has dropped to half. This can be ascertained by known methods, for example trimetrically. Afterwards, the other component is then added, with the amounts of isocyanate groups and OH or amino groups again being chosen to be equimolar. The reaction is continued until the isocyanate value has dropped to zero.

The copolymers obtained in this way are water-soluble or water-dispersible. Depending on the prepolymers used, the molecular weights M_{w} can be 500 to 250,000 g/mol, preferably 500 to 20,000 g/mol. The molecular weights can be determined by means of gel chromatography.

### Applications:

The copolymers to be used according to the invention can in principle be used in all fields where substances which are insoluble or only sparingly soluble in water are either to be used in aqueous preparations or are to develop their effect in an aqueous medium. Accordingly, the copolymers are used as solubilizers of substances which are sparingly soluble in water, in particular biologically active substances.

According to the invention, the term “sparingly soluble in water” also comprises virtually insoluble substances and means that, for a solution of the substance in water at 20° C., at least 30 to 100 g of water per g of substance are required. In the case of virtually insoluble substances, at least 10 000 g of water per g of substance are required.

For the purposes of the present invention, biologically active substances which are sparingly soluble in water are understood as meaning pharmaceutical active ingredients for humans and animals, cosmetic or agrochemical active ingredients or food supplements or dietetic active ingredients.

In addition, suitable sparingly soluble substances to be solubilized are also dyes, such as inorganic or organic pigments.

By virtue of the present invention, amphiphilic compounds in particular for use as solubilizers for pharmaceutical and cosmetic preparations and also for food preparations are provided. They have the property of solubilizing sparingly soluble active ingredients in the field of pharmacy and cosmetics, sparingly soluble food supplements, for example vitamins and carotenoids, but also sparingly soluble active ingredients for use in crop protection compositions, and also veterinary medicine active ingredients.

### Solubilizers for Cosmetics:

According to the invention, the copolymers can be used as solubilizers in cosmetic formulations. For example, they are suitable as solubilizers for cosmetic oils. They have a good solubilizing ability for fats and oils, such as peanut oil, jojoba oil, coconut oil, almond oil, olive oil, palm oil, castor oil, soybean oil or wheatgerm oil or for essential oils, such as dwarf pine oil, lavender oil, rosemary oil, spruce needle oil, pine needle oil, eucalyptus oil, peppermint oil, sage oil, bergamot oil, turpentine oil, melissa oil, sage oil, juniper oil, lemon oil, anise oil, cardamom oil; peppermint oil, camphor oil etc. or for mixtures of these oils.

In addition, the polymers according to the invention can be used as solubilizers for UV absorbers which are insoluble or sparingly soluble in water, such as, for example, 2-hydroxy-4-methoxybenzophenone (Uvinul® M 40, BASF), 2,2′,4,4′-tetrahydroxybenzo-phenone (Uvinul® D 50), 2,2′-dihydroxy-4,4′-dimethoxybenzophenone (Uvinul® D49), 2,4-dihydroxybenzophenone (Uvinul® 400), 2′-ethylhexyl 2-cyano-3,3-diphenylacrylate (Uvinul® N 539), 2,4,6-trianilino-p-(carbo-2′-ethylhexyl-1′-oxy)-1,3,5-triazine (Uvinul® T 150), 3-(4-methoxybenzylidene)camphor (Eusolex® 6300, Merck), 2-ethylhexyl N,N-dimethyl-4-aminobenzoate (Eusolex® 6007), 3,3,5-trimethylcyclohexyl salicylate, 4-isopropyldibenzoylmethane (Eusolex® 8020), 2-ethylhexyl p-methoxycinnamate and 2-isoamyl p-methoxycinnamate, and mixtures thereof.

The present invention therefore also provides cosmetic preparations which comprise at least one of the copolymers according to the invention of the composition specified at the start as solubilizers. Preference is given to those preparations which, besides the solubilizer, comprise one or more sparingly soluble cosmetic active ingredients, for example the abovementioned oils or UV absorbers.

These formulations are solubilizates based on water or water/alcohol. The solubilizers according to the invention are used in the ratio from 0.2:1 to 20:1, preferably 1:1 to 15:1, particularly preferably 2:1 to 12:1 relative to the sparingly soluble cosmetic active ingredient.

The content of solubilizer according, to the invention in the cosmetic preparation is, depending on the active ingredient, in the range from 1 to 50% by weight, preferably 3 to 40% by weight, particularly preferably 5 to 30% by weight.

In addition, further auxiliaries can be added to this formulation, for example nonionic, cationic or anionic surfactants, such as alkyl polyglycosides, fatty alcohol sulfates, fatty alcohol ether sulfates, alkanesulfonates, fatty alcohol ethoxylates, fatty alcohol phosphates, alkylbetaines, sorbitan esters, POE sorbitan esters, sugar fatty acid esters, fatty acid polyglycerol esters, fatty acid partial glycerides, fatty acid carboxylates, fatty alcohol sulfosuccinates, fatty acid sarcosinates, fatty acid isethionates, fatty acid taurinates, citric acid esters, silicone copolymers, fatty acid polyglycol esters, fatty acid amides, fatty acid alkanolamides, quaternary ammonium compounds, alkylphenol oxethylates, fatty amine oxethylates, cosolvents, such as ethylene glycol, propylene glycol, glycerol etc.

Further constituents which may be added are natural or synthetic compounds, e.g. lanolin derivatives, cholesterol derivatives, isopropyl myristate, isopropyl palmitate, electrolytes, dyes, preservatives, acids (e.g. lactic acid, citric acid).

These formulations are used, for example, in bath additive preparations such as bath oils, aftershaves, face tonics, hair tonics, eau de Cologne, eau de toilette and in sunscreen compositions. A further field of use is the oral care sector, for example in mouthwashes, toothpastes, adhesive creams for dentures and the like. In addition, the copolymers are also suitable for industrial applications, for example for preparations of sparingly soluble colorants, in toners, preparations of magnetic pigments and the like.

### Description of the Solubilization Method:

In the preparation of the solubilizates for cosmetic formulations, the copolymers according to the invention can be used as 100% strength substance or preferably as aqueous solution.

Usually, the solubilizer is dissolved in water and vigorously mixed with the sparingly soluble cosmetic active ingredient to be used in each case.

However, it is also possible to vigorously mix the solubilizer with the sparingly soluble cosmetic active ingredient to be used in each case and then to add demineralized water with continuous stirring.

### Solubilizers for Pharmaceutical Applications:

The claimed copolymers are likewise suitable for use as solubilizer in pharmaceutical preparations of any type which are notable for the fact that they can comprise one or more medicaments which are insoluble or sparingly soluble in water, and also vitamins and/or carotenoids. In particular, these are aqueous solutions or solubilizates for oral application.

Thus, the claimed copolymers are suitable for use in oral administration forms such as tablets, capsules, powders, solutions. Here, they can make the sparingly soluble medicament available with increased bioavailability. Particular preference is given to using solid solutions of active ingredient and solubilizer.

In the case of parenteral application, it is also possible to use emulsions, for example fatty emulsions, besides solubilizates. The claimed copolymers are also suitable for this purpose, in order to process a sparingly soluble medicament.

Pharmaceutical formulations of the abovementioned kind can be obtained by processing the claimed copolymers with pharmaceutical active ingredients by conventional methods and With the use of known and novel active ingredients.

The use according to the invention can additionally comprise pharmaceutical auxiliaries and/or diluents. Cosolvents, stabilizers, preservatives are especially mentioned as auxiliaries.

The pharmaceutical active ingredients used are substances which are insoluble or slightly soluble in water. According to DAB 9 (German Pharmacopoeia), the solubility of pharmaceutical active ingredients is categorized as follows: slightly soluble (soluble in 30 to 100 parts of solvent); sparingly soluble (soluble in 100 to 1000 parts of solvent); virtually insoluble (soluble in more than 10 000 parts of solvent). The active ingredients can here come from any area of indication.

Examples which may be mentioned here are benzodiazepines, antihypertensives, vitamins, cytostatics, in particular taxol, anesthetics, neuroleptics, antidepressants, antibiotics, antimycotics, fungicides, chemotherapeutics, urologics, thrombocyte aggregation inhibitors, sulfonamides, spasmolytics, hormones, immunoglobulins, sera, thyroid therapeutic agents, psychopharmacological agents, antiparkinsonians and other antihyperkinetic agents, ophthalmics, neuropathy preparations, calcium metabolism regulators, muscle relaxants, narcotics, antilipemics, hepatic therapeutic agents, coronary agents, cardiacs, immunotherapeutics, regulatory peptides and their inhibitors, hypnotics, sedatives, gynecological agents, antigouts, fibrinolytic agents, enzyme preparations and transport proteins, enzyme inhibitors, emetics, circulation-promoting agents, diuretics, diagnostics, corticoids, cholinergics, bile duct therapeutics, antiasthmatics, broncholytics, beta-receptor blockers, calcium antagonists, ACE inhibitors, antiarteriosclerotics, anti-inflammatories, anticoagulants, antihypotensives, antihypoglycemics, antihypertonics, antifibrinolytics, antiepileptics, antiemetics, antidotes, antidiabetics, antiarrhythmics, antianemics, antiallergics, anthelmintics, analgesics, analeptics, aldosterone antagonists and slimming agents.

One possible preparation variant is to dissolve the solubilizer in the aqueous phase, if appropriate with gentle heating and then to dissolve the active ingredient in the aqueous solubilizer solution. The simultaneous dissolution of solubilizer and active ingredient in the aqueous phase is likewise possible.

The use of the copolymers according to the invention as solubilizer can, for example, also be carried out by dispersing the active ingredient in the solubilizer, if appropriate with heating, and mixing it with water with stirring.

In addition, the solubilizers can also be processed in the melt with the active ingredients. In particular, solid solutions can be obtained in this way. Of suitability for this is, inter alia, also the melt extrusion process. Another way of preparing solid solutions is also to prepare solutions of solubilizer and active ingredient in suitable organic solvents and then to remove the solvent by customary methods.

The invention thus also generally provides pharmaceutical preparations which comprise at least one of the copolymers according to the invention as solubilizer. Preference is given to those preparations which, besides the solubilizer, comprises a pharmaceutical active ingredient which is insoluble or sparingly soluble in water, for example from the abovementioned areas of indication.

Of the abovementioned pharmaceutical preparations, particular preference is given to those which are orally applicable formulations.

The content of solubilizer according to the invention in the pharmaceutical preparation is, depending on the active ingredient, in the range from 1 to 75% by weight, preferably 5 to 60% by weight, particularly preferably 5 to 50% by weight.

A further particularly preferred embodiment refers to pharmaceutical preparations in which the active ingredients and the solubilizer are present as solid solution. Here, the weight ratio of solubilizer to active ingredient is preferably from 1:1 to 4:1.

### Solubilizers for Food Preparations:

Besides the use in cosmetics and pharmacy, the copolymers according to the invention are also suitable as solubilizers in the food sector for nutrients, auxiliaries or additives which are insoluble or sparingly soluble in water, such as, for example, fat-soluble vitamins or carotenoids. Examples which may be mentioned are clear drinks colored with carotenoids.

### Solubilizers for Crop Protection Preparations:

The use of the copolymers according to the invention as solubilizers in agrochemistry can comprise, inter alia, formulations which comprise pesticides, herbicides, fungicides or insecticides, especially also those preparations of crop protection compositions which are used as spray mixtures or pouring mixtures.

The block copolymers according to the invention are notable for a particularly good solubilizing effect.

The examples below illustrate the preparation and use of the block copolymers according to the invention in more detail.

### Preparation of the Vinyllactam Polymer

VP: N-Vinylpyrrolidone;

### General Procedure

Initial charge: 19.8 kg of feed 1, 880 kg of water

Feed 1: 360 kg of VP, 36 kg of mercaptoethanol

Feed 2: 3.1 kg, 27.9 kg of water

Feed 3: 1.8 kg 16.2 kg of water

For precise quantities see table below.

The preparation was carried out in a stirred reactor under a nitrogen atmosphere. The initial charge was heated to 80° C. at a stirrer speed of 60 rpm, admixed with 1.6 kg of feed, 2 g of feed 2 and polymerized for 15 min. The remaining amounts of feed 1 and feed 2 were added over a period of 2 hours. Then, feed 3 was added as a batch and the mixture was afterpolymerized for 3 hours at 80° C. The mixture was then cooled to 30° C. and the polymer was isolated by spray-drying.

The K value, measured at 1% strength by weight in water, was 12. The hydroxyl number was determined as described in Römpp Chemie Lexikon, 9th edition and was 65 mg KOH/g.

Determination of the molecular weight of the vinyllactam polymer by means of gel permeation chromatography gave Mₙ=720 g/mol, M_{w}=3560 g/mol, M_{w}/Mₙ=4.9. The determination was carried out with dimethylacetamide+0.5% by weight of LiBr as solvent (temperature: 80° C., flow rate: 1 ml/min, concentration of the solution: 5 g/l). The GPC column was calibrated using a suitable PMMA standard.

### Preparation of the Block Copolymers:

### EXAMPLES 1 TO 9

### General Procedure

25 mmol of isophorone diisocyanate, 200 g of solvent and 5 mmol of dibutyltin dilaurate were initially introduced and brought to the desired temperature. Then, 25 mmol, based on OH groups, of the polyalkylene oxide were added and the mixture was maintained at the set temperature until the isocyanate value had dropped to 50%. Then, 25 mmol, based on OH groups, of the vinyllactam prepolymer, were added and the temperature was increased to the chosen reaction temperature.

The OH numbers of the polyalkylene oxides and vinyllactam prepolymers are determined by acetylation of the hydroxyl groups with acetic anhydride and subsequent titration of the resulting acetic acid with base (DIN 53240 and DIN 16945, see Römpp, 9th edition).

The isocyanate value was determined titrimetrically: 1 g of the product was dissolved in 20 ml of a 0.1 molar solution of dibutylamine in toluene, and back-titrated with 0.1 molar hydrochloric acid using bromophenol blue as indicator.

| Ex. No. | Polyalkylene oxide used |
|---|---|
| 1 | Pluriol A 2000 E |
| 2 | Pluriol A 750 E |
| 3 | Pluriol P 2000 |
| 4 | Pluronic PE 3100 |
| 5 | Kerocom 3271 |
| 6 | Pluriol A 1000 PE |
| 7 | Pluronic PE 6100 |
| 8 | Pluronic PE 6200 |
| 9 | Pluronic PE 10100 |

| Example | Composition in mol %, based on use amount | | | |
|---|---|---|---|---|
| No. | VP | IPDI | Mercaptoethanol | AlkO |
| 1 | 12.73 | 1.82 | 1.82 | 83.63 |
| 2 | 26.92 | 3.85 | 3.85 | 65.38 |
| 3 | 26.92 | 3.85 | 3.85 | 65.38 |
| 4 | 37.84 | 5.41 | 5.41 | 51.43 |
| 5 | 41.18 | 5.88 | 5.88 | 47.06 |
| 6 | 25.01 | 3.57 | 3.57 | 67.84 |
| 7 | 29.79 | 4.25 | 4.25 | 61.71 |
| 8 | 24.56 | 3.51 | 3.51 | 68.42 |
| 9 | 19.72 | 2.82 | 2.82 | 74.64 |

| | | |
|---|---|---|
| (AlkO) | | |
| Pluriol | A750 E | 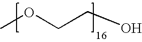 |
| | A 1000PE | 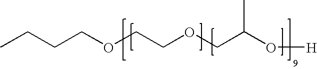 |
| | A 2000 E | 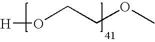 |
| | P 2000 | 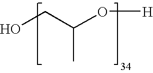 |
| Pluronic | PE 3100 | 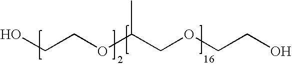 |
| | PE 6100 | 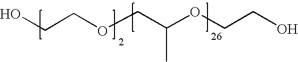 |
| | PE 6200 | 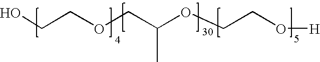 |
| | PE 10100 | 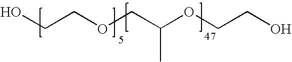 |
| Kero-com | 3271 | 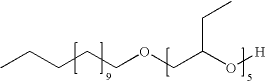 |

### Preparation of Solubilizates

2 g of the copolymer were weighed into a beaker. Then, one medicament in each case was weighed into the mixture as follows in order to obtain a supersaturated solution. (If the weighed-in mass dissolved in the medium, the initial weight was increased until a sediment formed).

Amount of active ingredient added: 17-β-estradiol 0.2 g; piroxicam 0.2; clotrimazol 0.2 g; carbamazepine 0.3 g; ketoconazole 0.25 g; griseofulvin 0.25 g; cinnarizine 0.25 g.

Phosphate buffer pH 7.0 was then added until solubilizer and phosphate buffer were present in the weight ratio 1:10. Using a magnetic stirrer; this mixture was stirred at 20° C. for 72 hours. There then followed a resting time of at least one hour. Following filtration of the mixture, it was measured photometrically and the content of active ingredient was determined.

| | Copolymer as in Ex. No./Solubilization at 20° C. in [g/100 ml] | | | | | | | | |
|---|---|---|---|---|---|---|---|---|---|
| Carbamazepine | 1/0.07 | 2/0.09 | 3/0.10 | 4/0.12 | 5/0.09 | 6/0.071 | 7/0.109 | 8/0.06 | 9/0.13 |
| Estradiol | | | 3/0.17 | 4/0.13 | 5/0.07 | | | | |
| Piroxicam | 1/0.17 | 2/0.31 | 3/0.18 | 4/0.24 | 5/0.24 | 6/0.18 | 7/0.26 | 8/0.22 | 9/0.23 |
| Clotrimazole | | | 3/0.22 | 4/0.13 | 5/0.13 | | | | |

1. A composition comprising polyvinyllactam-polyalkylene oxide block copolymers as solubilizers for substances which are sparingly soluble in water.

2. The composition according to claim 1, wherein the block copolymers are prepared by coupling polyvinyllactams that are terminated at one or both chain ends with hydroxyl groups with polyalkylene oxides via reaction with diisocyanates.

3. The composition according to claim 1 wherein the polyvinyllactams have K values of from 6 to 20.

4. The composition according to claim 1 wherein the polyvinyllactam used is polyvinylpyrrolidone.

5. The composition according to claim 1 wherein the polyalkylene oxides have molecular weights of from 300 to 10,000 daltons.

6. The composition according to claim 1 wherein the polyalkylene oxides are chosen from the group consisting of polyethylene glycols, polypropylene glycols, polytetrahydrofurans, polybutylene glycols and the monoalkoxylated derivatives of said polyalkylene oxides.

7. The composition according to where claim 1 wherein the polyalkylene oxides used are polyoxyethylene-polyoxypropylene block copolymers.

8. The composition according to where claim 1 wherein the coupling of the polyalkylene oxide side chains takes place via diisocyanates.

9. The composition according to claim 1 wherein the polyalkylene oxides are functionalized by reaction with a diisocyanate prior to the reaction with the vinyllactam copolymer.

10. The composition according to claim 1, wherein the polyalkylene oxides are used in equimolar amounts, based on the hydroxyl groups of the vinyllactam copolymer.

11. The composition according to claim 1 wherein the diisocyanate used is isophorone diisocyanate.

12. The composition according to claim 1, wherein the polyvinyllactam-polyalkylene oxide block copolymers have an A-B, A-B-A or B-A-B structure.

13. The composition according to claim 1, wherein the copolymers have molecular weights M_{w} of from 700 to 20,000 g/mol.

14. The composition according to claim 1, wherein the substances which are sparingly soluble in water are biologically active substances.

15-20. (canceled)

21. A preparation of substances which are sparingly soluble in water, obtainable using polyvinyllactam-polyalkylene oxide block copolymers as solubilizers according to claim 1.

22. The preparation according to claim 22, comprising a biologically active substance as a substance which is sparingly soluble in water.

23. The preparation according to claim 22, comprising a pharmaceutical active ingredient as a biologically active substance which is sparingly soluble in water.

24. The preparation according to claim 23 in the form of orally applicable administration forms.

25. The preparation according to claim 21, comprising a cosmetic active ingredient as a biologically active substance which is sparingly soluble in water.

26. The preparation according to claim 21, comprising an agrochemical active ingredient as a biologically active substance which is sparingly soluble in water.

27. The preparation according to claim 21, comprising a food supplement or a dietetic active ingredient as a biologically active substance which is sparingly soluble in water.

28. The preparation according to claim 21, comprising a dye as a substance which is sparingly soluble in water.

29. A pharmaceutical preparation for the treatment of illnesses comprising the composition according to claim 1.

30. A cosmetic preparation comprising the composition according to claim 1.

31. An agrochemical preparation comprising the composition according to claim 1.

32. A food supplement or dietetic agent comprising the composition according to claim 1.

33. A food comprising the composition according to claim 1.

34. A dye preparation comprising the composition according to claim 1.
